(19) United States
(12) Patent Application Publication
(10) Pub. No.: US 2005/0079508 A1
(43) Pub. Date: Apr. 14, 2005

(54) Constraints-based analysis of gene expression data

(76) Inventors: Judy Dering, Westlake Village, CA (US); Cindy A. Wilson, Santa Monica, CA (US); Dennis Slamon, Woodland Hills, CA (US)

Correspondence Address: GREENBERG TRAURIG LLP, 2450 COLORADO AVENUE, SUITE 400E, SANTA MONICA, CA, 90404, US

(21) Appl. No.: 10/683,535

(22) Filed: Oct. 10, 2003

(51) Int. Cl. C12Q 1/68; G06F 19/00; G01N 33/48; G01N 33/50

(52) U.S. Cl. 435/6; 702/20

(57) ABSTRACT

In accordance with one aspect of the invention, a constraints-based method of analysis of gene expression data has been shown to provide a useful method for target identification in gene expression profiles. Such constraints-based methods of analysis provide useful analytical methods that focus on genes and related pathways that are likely to be important in disease progression, that is, to provide likely candidates that will serve as targets for various therapeutics.

The present invention generally relates to gene expression data analysis. More particularly and in one aspect of the invention, the teachings disclosed herein provide novel methods for constraints-based analysis of gene expression data.

### BACKGROUND OF THE INVENTION

Cancer is a heterogeneous disease in most respects, including its cellularity, different genetic alterations and diverse clinical behaviors. Many analytical methods have been used to study human tumors and to classify samples into homogeneous groups that can predict clinical behavior. DNA microarrays have made significant contributions to this field by detecting similarities and differences among tumors through the simultaneous analysis of expression of thousands of genes.

Gene expression data are often referred to as “signatures” or “portraits,” because most tumors showed special patterns that are unique and recognizable. Coupled with statistical analysis, DNA microarrays have allowed investigators to develop expression-based classifications for many types of cancer including breast, brain, ovary, lung, kidney, and lymphoma. Portraits/signatures of those in a “malignant family” may seem different from one another, but they all have features that are common to their family and that differentiate them from members of a “benign family.” Some functional classes of genes are invariably altered when normal cells transform to malignant, including genes involved in cell-cycle control, adhesion and motility, apoptosis and angiogenesis. Thus, despite the morphological and molecular heterogeneity among different cancers types, there are common threads that allow members to be recognized as branches of the same family tree.

The main challenge to the study and treatment of cancer is resolving the tumor heterogeneity that exists both between and within tumor types. By light microscopy, the cellular complexity of a tumor can be visually dissected through differences in the appearance of malignant and nonmalignant cells. By using microarrays, the makeup of complex tissue samples can be resolved as dominant patterns of gene expression representing the origin and function of different cell types. For example, solid tumors can be molecularly dissected into epithelial cells, infiltrating lymphocytes, adipose cells and surrounding stromal cells. Microarray analysis can do more than differentiate a mixture of cell types and can often resolve levels of heterogeneity that are not apparent by eye. Because the clinical behavior of tumors cannot be accounted for completely by morphology, it is the hope of medicine that molecular taxonomy based on “signature” profiles will provide a more accurate prognosis and prediction of response to therapy.

The analysis of microarray data obtained from tumor samples is extremely complex. There are two general, prior art statistical approaches for tumor classification. The first is “supervised” analysis in which one searches for genes whose expression patterns correlate with an external parameter. Most commonly used supervising parameters are clinical features such as patient survival, presence of metastases and response to therapy. Many statistical metrics have been used successfully in “supervised” analyses including the standard t-test and signal-to-noise ratios.

Algorithms such as weighted voting, K-nearest neighbor classifiers, support vector machines and artificial neural networks can be applied to a set of genes selected using one of these metrics to build models capable of predicting the class a particular tumor sample. To test the robustness of classification, these methods are often coupled with a leave-one-out cross validation analyses in which one of the samples from the original “training” set is withheld and a class prediction is made on the withheld sample.

A second approach is “unsupervised” analysis, in which no external feature is used to guide the analysis process. Instead, the data are used to search for patterns without any a priori expectation concerning the number or type of groups that are present.

The most common “unsupervised” analysis method is hierarchical cluster analysis. Each analytical method has its own strengths and weaknesses and because classifications tend not to be mutually exclusive, most investigators base the significance of their microarray findings on more than one analysis.

Although both methods can analyze the expression of thousands of genes, minimization of a discriminatory gene list can ease the biological interpretation and facilitate use in clinical tests. Several methods have been used for gene selections such as correlation metrics or t-test coupled with permutation testing. Other methods work by selecting a gene list that gives rise to the highest prediction accuracy during leave-one-out cross-validation or nearest “centroid” (Tibshirani et al., 2002.) analysis.

Gene expression profiling has been utilized to predict the clinical outcome of breast cancer patients, that is, to identify a gene expression signature or portrait that can be associated with disease outcome. Signatures can also be associated with histopathological data, such as estrogen receptor expression as determined by immunohistochemical staining (van t Veer et al., 2002). In one study, DNA microarray analysis of primary breasts tumors of patients and application of supervised classification to identify a gene expression signature that strongly predicted a short interval to distant metastasis otherwise known as a poor prognosis signature, was utilized for patients without tumor cells in local lymph nodes at diagnosis. The poor prognosis signature consists of genes regulating cell cycle, invasion, metastasis and angiogenesis. This gene expression profile was utilized as a critical parameter for predicting disease outcome. In this same study, an unsupervised hierarchical clustering algorithm was used to cluster tumors on the basis of similarities measured over approximately 5000 genes that were identified as having significant regulation across 98 primary breast cancer tumors. Unsupervised clustering provides for some extent of distinction between “good prognosis” and “bad prognosis” tumors.

To identify reliably good and poor prognostic tumors, a three-step supervised classification method was used in the van t Veer et al. study, where firstly approximate 5000 genes significantly regulated in more than 3 tumors out of 78 were selected from the 25,000 genes on the microarray. The correlation coefficient of expression for each gene with disease outcome was calculated and 231 genes were found to be significantly associated with disease outcome (correlation coefficient, <−0.3 or >0.3). In a second step, these 231 genes were rank ordered on the basis of magnitude of the correlation coefficient. Third, the number genes in the “prognosis classifier” were optimized by sequentially adding subsets of five genes from the top of this rank-ordered list and evaluating its power for correct classification using the leave-one-out method for cross-validation. Classification here was made on the basis of the correlation of the expression profile of the leave-one-out sample with the mean expression levels of the remaining samples from the good and the poor prognosis patients respectively. The accuracy improved until an optimal number of marker genes was reached (70 genes).

In two-dimensional cluster analysis, gene clustering and tumour clustering are performed independently using an agglomerative hierarchical clustering algorithm. For gene clustering, pairwise similarity metrics among genes are calculated on the basis of expression ratio measurements across all tumours. Similarly, for tumour clustering, pairwise similarity measures among tumours are calculated based on expression ratio measurements across all significant genes.

The method for classifying breast tumours into prognostic or diagnostic categories based on gene expression profiles developed by van t Veer et al. includes the following three steps: (1) selection of discriminating candidate genes by their correlation with the category; (2) determination of the optimal set of reporter genes using a leave-one-out cross validation procedure; (3) prognostic or diagnostic prediction based on the gene expression of the optimal set of reporter genes.

In another study (Perou et al., 2000), variations of gene expression patterns of a set of 65 surgical specimens of human breast tumors from 42 different individuals using DNA microarrays representing 8102 human genes were characterized, each array providing a distinct molecular portrait of each tumor. The tumors were classified into subtypes distinguished by their gene expression patterns. That is, the phenotypic diversity observed in these breast tumors were accompanied by a corresponding diversity in gene expression patterns that could be captured using cDNA microarrays. Pools of mRNA isolated from different cultured cell lines provided a common reference sample and internal standard against which the gene expression of each experimental sample was compared. In this study, a hierarchical clustering method was used to group genes on the basis of similarity in the pattern with which their expression varied over all samples. The same clustering method was used to group the experimental samples on the basis of similarity in their gene pattern expression. The hierarchical clustering algorithm used in this study organizes the experimental samples only on the basis of overall similarity in their gene expression patterns. In a later work by the same group (Sorlie et al., 2001) hierarchical clustering methods were utilized to further refine previous classifications of gene expression patterns from tumors by analyzing a larger number of tumors and further exploring the clinical value of subtypes/classification of tumors based upon their gene expression patterns.

However, constraints-based analysis of gene expression data, particularly public expression data and cell line data, has not yet been undertaken. Such gene expression data has been typically analyzed utilizing the above mentioned clustering approaches. These clustering approaches assume that groups are unknown at the start of the investigation and need to be determined. Alternatively, constraints-based analysis “constrains” samples into groups on the basis of some predefined characteristic or set of characteristics, and then investigates gene expression patterns among these groups. Additionally, identification of overexpression of ROR1, an orphan receptor tyrosine kinase, in cancer cell lines and tumors, has not been identified or utilized as a marker which can be utilized in cancer prognosis.

### SUMMARY OF THE INVENTION

Now in accordance with one aspect of the invention, there has been found that a constraints-based method of analysis of gene expression data provides a useful way for target identification in gene expression profiles. Such constraints-based methods of analysis provide useful methods to focus on genes and related pathways that are likely to be important in disease progression, that is, to provide likely candidates that will serve as a target for various therapeutics.

In one example, such constraints-based methods can be applied to a particular disease. For example, breast cancer is one disease for which an abundance of information (from experimental studies as well as gene expression profiles) is available and thus lends itself to such constraints-based analysis as taught by the present invention.

Another aspect of the present invention relates to constraints-based analysis of public expression data that is typically obtained from microarray studies of various tissue samples. In one embodiment, a working gene set defined by the expression of receptor tyrosine kinases (RTK) and associated ligands is investigated.

In still another aspect of the present invention, tumor data samples and RTK gene sets are subjected to subtype grouping based on predefined biological constraints to reveal biologically relevant differences in gene expression that can then be statistically verified.

In one aspect of the invention, a constraints-based method for identifying a genomic target of interest from gene expression profiles comprises obtaining tissue sample expression data sets and first selecting a working gene-expression set, the gene-expression set having a plurality of members, second defining subgroups of the tissue samples of the expression data sets, wherein the subgroup definition is a constrained definition, and third analyzing co-expression of the members of the working gene set across the subgroups to identify potential gene targets.

In one embodiment, the working gene expression set can comprise at least one receptor tyrosine kinase and/or a receptor and a ligand.

In some examples, tissue sample expression data sets are comprised of expression data sets from tumor samples. In additional examples, the tissue sample expression data sets are comprised of expression data sets from tissue from a mammal. In still other examples, the tissue sample expression data sets are comprised of expression data sets attained tissue from at least one of a human, mouse, primate, canine, pig, rat, and feline.

In some embodiments, the methods provided by the teachings of the invention can be utilized for analysis comprised of expression data sets based upon embryonic tissue and/or any gene expression sample expression data set.

Tumor expression data to be analyzed according to the teachings of the present invention can be malignant tumors or benign tumors from any origin, including without limitation, from breast, liver, digestive tract, etc.

The constraints-based data analysis of the present invention may further comprise a step of selecting known prognostic markers that are correlative with prognostic outcomes as either part of the working gene set or one of the set of characteristics that define groups

In another aspect of the present invention, use of an orphan receptor tyrosine kinase's (ROR1) overexpression may be utilized as a prognostic marker in cancer patients, more particularly, as a marker associated with a poor prognosis.

### BRIEF DESCRIPTION OF THE FIGURES

The foregoing aspects and many of the attendant advantages of this invention will become more readily appreciated as the same becomes better understood by reference to the following detailed description, when taken in conjunction with the accompanying figures, wherein:

FIG. 1 is a graph of ESR1 mRNA expression levels in various groups;

FIG. 2 is a graph of HER2 (or ERBB2) and the closely-linked GRB7 mRNA expression of the groups demonstrating gene amplification; and

FIG. 3 is a graph of ROR1 expression.

### DETAILED DESCRIPTION OF THE PREFERRED EMBODIMENTS

Particular embodiments of the invention are described below in greater detail for the purpose of illustrating its principles and operation. However, various modifications may be made, and the scope of the invention is not limited to the exemplary embodiments or operations described. For example, while specific reference is made to ROR1 identification, it can be appreciated that any gene's overexpression in various samples may be detected and quantified in accordance with the teachings of the present invention. Likewise, the teachings of the present invention are accordingly applicable to diseases or conditions other than cancer, for which appropriate gene expression data is available, as Recognizable to those of ordinary skill in the art.

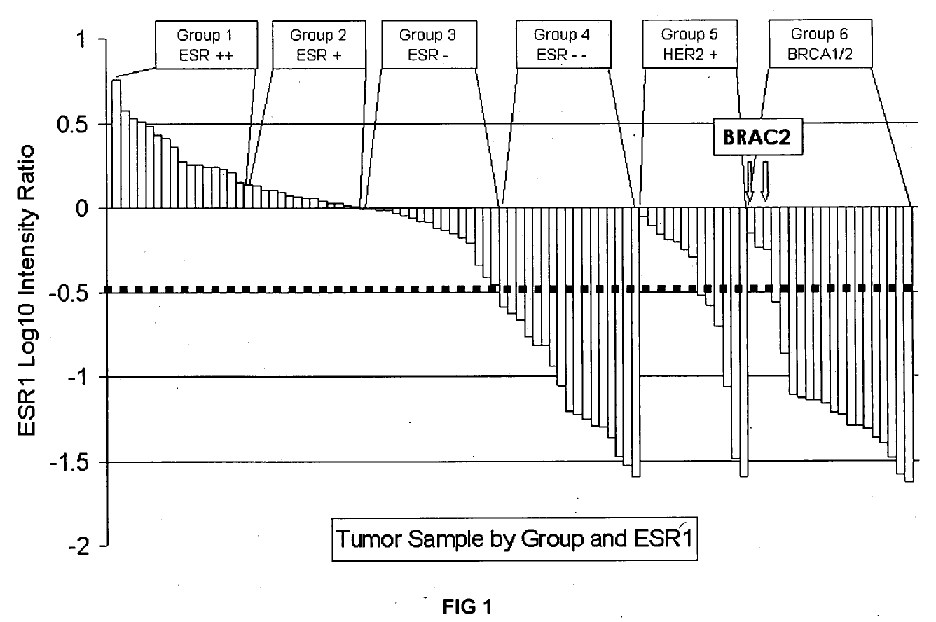

One of the initial steps for approaching the public expression data for analysis is the definition of selection of constraints or criteria which will determine tumor classification. These classifications can then be applied to publicly available microarray data sets, such as the Rosetta/Netherlands prognosis study (van't Veer 2002). The primary finding of the van't Veer study was the identification of a poor prognosis signature or profile that identified 70 genes that predict poor prognosis within 83% accuracy. The relevance of the 70 genes identified to the biology of rapid metastasis is unclear. Twenty-nine of these seventy genes are of unknown function. Previously described prognostic genes, such as HER-2, ER-α, cyclinD1, UPA/PAI-1 were not part of the signature. No novel receptor tyrosine kinase was identified as a prognostic marker or potential target in this study. In particular, the receptor tyrosine kinase ROR1 was not associated with a poor prognosis group. Although unsupervised two-dimensional clustering was used on the 5000 differentially regulated genes across 98 tumors distinguished to some extent two groups of tumors with different prognosis, the 70 marker genes were actually identified in a three-step classification method. (van't Veer FIG. 1) In the first step, the correlation coefficient of each differentially regulated gene with disease outcome was calculated and 231 genes were identified as significantly regulated. In step two, these 231 genes were rank-ordered based on the absolute value of the correlation coefficient. In step three, the number of genes in the classifier was optimized by sequentially adding subsets of 5 genes from the top of rank-ordered list and evaluating the accuracy of the classification using the leave-one-out method. The supervised hierarchical clustering method is used on the 70 prognostic markers to illustrate the expression patterns associated with the two prognostic groups identified (van't Veer 2002).

In a study by Sorlie et al. (2001), primary findings include the identification of five subtypes of breast carcinoma associated with significantly different clinical outcomes. The microarrays used in these studies included 8,102 genes, but did not include probes for many receptor tyrosine kinases and other oncogenes. Specifically, the receptor tyrosine kinase ROR1 was not represented on the microarray. The authors used the statistical method SAM (significance analysis of microarrays) to identify the a subset of genes associated with prognosis. This “intrinsic gene list” was used as the basis for classification and cluster analysis. 78 carcinomas and seven nonmalignant breast samples were analyzed across the intrinsic gene list using an unsupervised hierarchical clustering technique. (Sorlie et al.) The authors identify either 5 or 6 subgroups, depending on the tumor samples included in the classification. These subgroups have different rates of mutation of the TP53 gene, as well as different prognosis. The authors identify marker genes associated with each group on the basis of their expression patterns.

Both van't Veer and Sorlie utilize statistical methods to select the gene set used in clustering and classification. Both methods are based on correlation of gene expression to prognosis, and do not give special weight or attention to prognostic markers and molecules that have been shown to be important (e.g. overexpressed) in subtypes of breast cancers, for example estrogen receptor (ESR1) and the human epidermal growth factor receptor-2, (HER2 or ERBB2). According to the teachings of the present invention, constraints-based hypothesis building focuses on genes and pathways likely to be important for disease progression. Accordingly, a constraints-based method for target identification in gene expression profiles is developed and provided herein.

A first step in the method of the present invention is to select a working gene set comprising molecules and related family members identified as potentially important in the pathogenesis of a disease, here and for example breast cancer. A working set of about 400 genes was defined. Genes previously associated with breast cancer were identified from review of the published literature as well as such sources as the Online Mendelian Inheritance in Man (OMIM), Breast Cancer Database and NCBI Nucleotide database. The complete class of receptor tyrosine kinases and their ligands was included, as well as genes known to be regulated by HER2 from the analysis of cell line data. (Slamon, unpublished data) Chemokines, adhesion molecules and epithelial junction proteins were also included. These genes were included in the study regardless of their correlation to prognosis in any specific data set.

In this example (the study of breast cancer), after the working gene set was selected, the expression pattern of the selected genes in the van't Veer data was investigated. (Because many of 400 genes were not included on the microarrays used in the Sorlie study, the constraints-based method was not applied to this data.) This entails downloading gene expression data files for about 25,000 genes for 78 sporadic and 20 breast cancer susceptibility (BRCA) tumors. These files were made available publicly by the van't Veer group. For each tumor sample, two hybridizations were made to microarrays containing 25,000 human genes, using a fluorescent dye reversal technique. Fluorescence intensities of scanned images of the microarray slides were quantified and normalized, and represent the transcript abundance of a gene as an intensity ratio of the sample signal to the signal of the reference pool. The reference pool was created from an equal amount of CRNA from each individual sporadic patient. Therefore, in this study each individual sample was compared to the “average” sample. All ratios were expressed in Log10 format in the van't Veer study. This ratio provides the basic level of analysis for the constraints-based method. The ratios for the 400 genes defined as part of the working set were extracted from the van't Veer data. A matrix was created with each row representing a gene, each column representing a sample, and the data values were the intensity ratios.

After selecting the genes, thresholds or “cutting values” were defined to create categories of gene expression levels. All ratios with a value greater than 0.25 (corresponding to approximately a 1.78 fold increase, Antilog₁₀(0.25)=1.78) were categorized as up-regulated, ratios less than −0.25 were categorized as down-regulated, and ratios between 0.25 and −0.25 were classified as normal expression.

After selecting working gene set and expression thresholds, criteria for determining tumor subtypes were defined and applied to the van't Veer data. As the Sorlie study demonstrates, there are tumor subtypes that are associated with different clinical outcome. Sorlie et al. (2001) determined the subtypes by unsupervised hierarchical clustering. In the constraints-based method, tumor samples were “binned” into groups rather than “clustered”. Binning divides the possible values for some observation into intervals, and then counts how many observations fall into each bin. The defined bins may or may not be of equal width. (Tukey, 1977) We selected three markers to act as constraints to bin tumors into groups; ESR1 expression level, HER2 (ERBB2) expression level and BRCA mutation status. (In this study, samples were classified as positive for BRCA if patients were carriers of a germline mutation in either the BRCA1 or BRCA2 gene.) The mRNA expression levels of ESR1 and HER2 are internal to the microarray data set, while the information about the BRCA mutation status was included as an external parameter as part of the clinical data for each patient. There is significant evidence in the literature that ESR1, HER2 and BRCA mutation status are associated both with prognosis and different pathogenic pathways.

Table 1 shows the distribution of the van't Veer samples across the breast tumor subgroups according to the defined categories, in accordance with the present invention, that is, application of constraints-based method to create breast tumor subgroups in Rosetta/Netherlands data. The sporadic tumor samples were first classified or binned on the basis of their HER2 expression. Those samples with HER2 (or ERBB2) expression ratio >0 were classified as HER2+. The remaining sporadic samples (all with HER2<0) were then grouped by their ESR1 mRNA expression. Four bins or categories based on the level of ESR1 expression were created. All samples with ESR1 intensity ratio >0.2 were classified as ESR1++ (highest ESR1 group). The interval between 0.2 and 0 defines or bins the ESR1+ group (moderate ESR1). The interval between 0 and −0.5 defined the ESR1− group (low ESR1). Finally, those samples with ESR1<−0.5 were defined as ESR−− group (lowest ESR1). Samples having BRCA mutations were classified as a separate group (Group 6). The HER2+ and ESR1−− tumors had the poorest prognosis, followed by ESR1++. Prognosis information was not available for the BRCA patients.

TABLE 1

| | | | | | | Known | | | |
|---|---|---|---|---|---|---|---|---|---|
| | | | | | | Prognosis | | | |
| | | Good prognosis | | Poor prognosis | | by Group | | | |
| Group | Group | | % | | % | | % | Total | |
| No. | Name | Samples | Group | Samples | Group | Samples | Total | Samples | Description |
| 1 | ESR1++ | 9 | 56% | 7 | 44% | 16 | 21% | 16 | ESR1 >= 0.2 |
| 2 | ESR1+ | 10 | 67% | 5 | 33% | 15 | 19% | 15 | 0.2 > ESR1 >= 0 |
| 3 | ESR1− | 12 | 71% | 5 | 29% | 17 | 22% | 17 | 0 > ESR1 >= −0.5 |
| 4 | ESR1−− | 6 | 35% | 11 | 65% | 17 | 22% | 17 | ESR1 < −0.5 & HER2 < 0 |
| 5 | HER2+ | 6 | 46% | 7 | 54% | 13 | 17% | 13 | HER2 > 0 |
| 6 | BRCA | N/A | | N/A | | N/A | | 19 | BRCA Mutation |
| | | | | | | | | | ESR1 < 0 & HER2 < 0 |
| | Total | 43 | 55% | 35 | 45% | 78 | | 97 | |

TABLE 2

| Rosetta/Netherlands Data | | | | | Stanford/Norway Data | |
|---|---|---|---|---|---|---|
| Group | Group | ESR1 | ERBB2 | BRCA | Group | |
| No. | Name | Ratio* | Ratio* | Mutation | Name | Markers |
| 1 | ESR1++ | >=0.2 | <0 | No | Luminal A | High levels of ESR1, GATA3, HNF3A, LIV-1 |
| 2 | ESR1+ | 0.0-0.2 | <0 | No | Luminal B | Moderate Levels of Luminal A |
| 3 | ESR1− | −0.5-0.0 | <0 | No | Luminal C | Low Levels of Luminal A |
| 4 | ESR1−− | <=−0.5 | <0 | No | Basal | High CDH3, KRT17, KRT5, FABP7 |
| 5 | HER2+ | <0 | >0 | No | ERBB2 | High ERBB2, GRB7, STARD3 |
| 6 | BRCA | <0 | <0 | Yes | N/A | |

| | |
|---|---|
| Constrained definition of classes based on expression | Cluster-based definition of classes. Markers are a subset of |
| level of ESR1 and ERBB2, as well as the identification | those selected by study authors as exemplars for clusters. |
| of a BRCA mutation. | |

*Expression levels are measures as Log10 intensity ratio of sample to reference.

Table 2 compares the subgroups defined in accordance with the present invention from the van't Veer (Rosetta/Netherlands) data with the clusters discovered in the Sorlie (Stanford/Norway) data. Expression levels in this table are measured as Log10 intensity ratios of sample to reference as described previously. There are similarities between the gene expression patterns of the Sorlie clusters discovered by unsupervised hierarchical clustering to the constrained groups. The ESR1++ group is similar to the Luminal A groups, and also has high levels of GATA3, HNF3A, and LIV-1. The ESR1−− group shows high levels of expression of the markers found in the Stanford/Norway basal group, i.e. CDH3, KRT17 and KRT5.

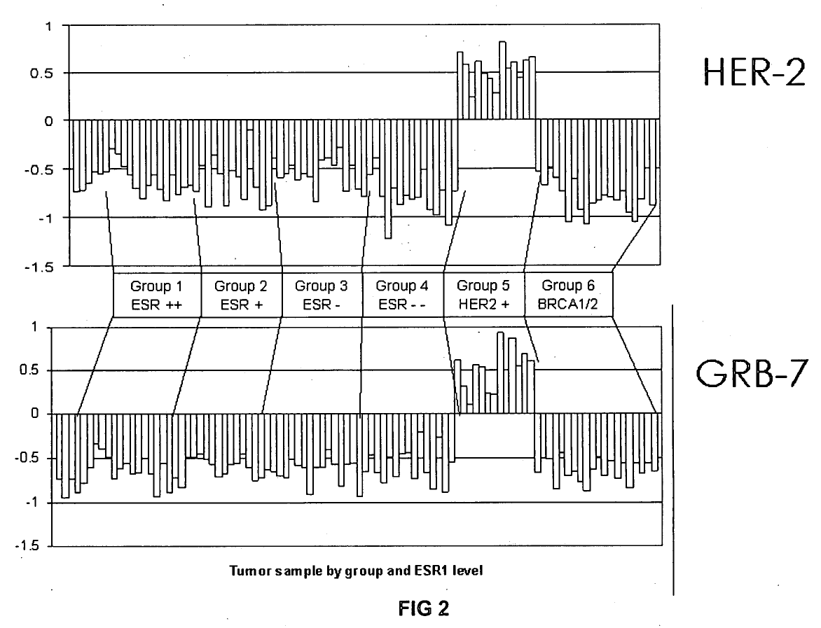

FIG. 1 graphs the level of ESR1 expression by tumor group and ESR1 level. As is evident from this graph, ESR1 expression is a continuous variable. As stated above, ESR1 expression was used to define groups 1-4. All of the samples in groups 5 (HER2+) & 6 (BRCA) have ESR1<0. This is a biological phenomenon, and not a matter of definition or constraints. This continuous expression can be contrasted to the expression of HER2 and GRB7 displayed in FIG. 2. Only Group 5, which was defined by having HER2 expression >0, shows positive expression levels of HER2 and GRB7. This is consistent with the fact that HER2 overexpression is the result of gene amplification. GRB7 is a gene positioned closely to HER2 on the 17q chromosome. Overexpression of GRB7 (as well as other genes that make up the HER2 amplicon) is consistently found with overexpression of HER2.

TABLE 3

| | | Sample | | | |
|---|---|---|---|---|---|
| Matrix | Data Level | Level | Gene Level | Built From | # Matrices |
| Level 1 - | Ratios | Sample | Gene | Downloaded Data | One per Group |
| Gene | | | | | |
| Level 2 - | Binary | Sample | Gene | Level 1 - Gene | Two per Group; |
| Gene | | | | | Up-regulation & Down-regulation |
| Level 3 - | Count | Group | Gene | Level 2 - Gene | Two; |
| Gene | | | | | Up-regulation & Down-regulation |
| Level 2 - | Binary | Sample | Gene Set | Level 2 - Gene | Two per group; |
| Gene Set | | | | | Up-regulation & Down-regulation |
| Level 3 - | Count | Sample | Gene Set | Level 2 -Gene Set | Two per group; |
| Gene Set | | | | | Up-regulation & Down-regulation |
| Level 4 - | Count | Group | Gene Set | Level 3 - Gene Set | Two per group; |
| Gene Set | | | | | Up-regulation & Down-regulation |
| Level 2 - | Binary | Sample | Gene Sets Union | Level 2 - Gene Set | Two per group; |
| Gene Set Union | | | | | Up-regulation & Down-regulation |
| Level 3 - | Count | Sample | Gene Sets Union | Level 2 - Gene Set | Two per group; |
| Gene Set Union | | | | | Up-regulation & Down-regulation |
| Level 4 - | Count | Group | Gene Sets Union | Level 2 - Gene Set Union | Two; |
| Gene Set Union | | | | | Up-regulation & Down-regulation |

TABLE 4

| | Sample # | | | | | | | | | | | | | | | | |
|---|---|---|---|---|---|---|---|---|---|---|---|---|---|---|---|---|---|
| | 7 | 8 | 12 | 20 | 24 | 28 | 44 | 48 | 50 | 57 | 65 | 67 | 68 | 71 | 73 | 75 | 77 |
| | Group # | | | | | | | | | | | | | | | | |
| | 4 | 4 | 4 | 4 | 4 | 4 | 4 | 4 | 4 | 4 | 4 | 4 | 4 | 4 | 4 | 4 | 4 |
| EGF | 0.11 | −0.05 | 0.57 | −0.03 | 0.23 | 0.36 | −0.14 | −0.24 | −0.15 | −0.13 | −0.46 | −1.46 | −0.21 | −0.28 | −0.16 | −0.27 | −0.16 |
| TGFA | −0.24 | −0.30 | 0.27 | 0.84 | 0.24 | 0.44 | 0.39 | −0.25 | 0.34 | 0.91 | 0.23 | −0.09 | 0.07 | −0.52 | 0.39 | 0.01 | −0.35 |
| AREG | −0.18 | −0.98 | −1.24 | −1.03 | −1.32 | 0.07 | −1.06 | −1.27 | −1.16 | −1.46 | −0.91 | −0.82 | −1.47 | −0.84 | −1.21 | −0.85 | −1.00 |
| BTC | −0.33 | −0.06 | 0.50 | −0.02 | −0.24 | 0.06 | 0.06 | 0.10 | 0.00 | 0.12 | 0.09 | 0.13 | 0.08 | −0.02 | 0.98 | 0.01 | −0.09 |
| EREG | 0.04 | −0.03 | 0.77 | −0.09 | −0.29 | 0.10 | −0.12 | −0.08 | −0.12 | −0.19 | −0.10 | 0.03 | −0.23 | −0.10 | 0.44 | −0.20 | −0.26 |
| NRG2(1) | −0.15 | 0.10 | 1.34 | −0.09 | −0.20 | 1.12 | −0.24 | −0.22 | 0.02 | 0.11 | 0.06 | 0.42 | 0.03 | −0.06 | 0.38 | −0.08 | −0.23 |
| NRG2(2) | 0.12 | −0.11 | 0.28 | −0.09 | 0.11 | 1.04 | −0.32 | −0.17 | −0.06 | −0.07 | 0.01 | 0.44 | 0.18 | −0.11 | 0.38 | −0.12 | −0.14 |
| NRG2(3) | 0.10 | −0.21 | 0.28 | −0.12 | 0.14 | 1.14 | −0.42 | −0.23 | −0.09 | −0.20 | −0.05 | 0.58 | 0.33 | −0.09 | 0.44 | −0.04 | −0.19 |
| EGFR | 0.00 | 0.03 | 0.03 | 0.09 | 0.03 | 0.12 | 0.21 | 0.12 | 0.28 | −0.27 | 0.15 | 0.39 | 0.08 | 1.29 | 0.33 | 0.10 | −0.03 |
| (ERBB1) | | | | | | | | | | | | | | | | | |
| ERBB2 | −0.40 | −0.78 | −0.73 | −0.70 | −0.78 | −0.57 | −0.72 | −0.80 | −0.93 | −1.09 | −0.98 | −0.51 | −1.23 | −0.82 | −0.79 | −0.87 | −0.71 |
| (HER2) | | | | | | | | | | | | | | | | | |
| ERBB3 | 0.07 | −0.28 | −0.44 | −0.68 | −0.52 | −0.13 | −0.03 | −0.51 | −0.19 | −0.74 | −0.38 | −0.23 | −0.69 | −0.35 | −0.41 | −0.48 | −0.24 |
| (HER3) | | | | | | | | | | | | | | | | | |
| ERBB4 | −0.02 | −0.28 | −1.56 | −0.54 | −1.08 | 0.08 | −1.02 | −0.75 | −0.69 | −2.00 | −0.70 | −0.69 | −0.30 | −0.26 | −0.65 | −0.43 | 0.29 |
| (HER4) | | | | | | | | | | | | | | | | | |

Level 1 Matrix - mRNA expression represented as Log10 Intensity Ratios Excerpt of the members of EGF family of Ligands and Receptors by Sample for Group 4 (ESR1−)

After samples have been constrained into groups and thresholds for expression levels have been defined, the frequency of up-regulated and down-regulated genes across individual samples and by groups can be investigated. Matrices are then created which provide the basis for the investigation of the coexpression of members of the working gene set across tumor groups, which in turn generates hypotheses regarding pathogenesis by tumor group. The data in the matrix is organized by sample level, gene level, and type of data value. There are two levels of analysis of samples: sample level 1 is across individual samples and sample level 2 is across tumor groups. There are three levels of analysis of genes: gene level 1 is by individual gene, gene level 2 is by gene set, and gene level 3 computes the co-expression (or union) of gene sets. The data values in the matrix are either intensity ratios, binary expression values based on defined thresholds, or counts of binary expression values. Table 3 is a table that shows how the data values, gene levels and sample levels are combined to build the various types of matrices used in the constraints-based method according to the present invention. Analyzing these matrices provides a method for identifying potential targets for therapies (for example, antibody, small molecules, drug..etc) that are then candidates for further experimental validation and can be tested/verified for statistical significance.

The focus, in this embodiment, was on receptor tyrosine kinases. The working genes set included ail receptor tyrosine kinases and their ligands that were available in Rosetta/Netherlands data ( the microarray contained 147 probes representing 127 out of 130 possible unique RTKs and their ligands.) This provides for identification of tumor group specific RTK/ligand expression. A list of the ligands and receptors that make up this class and are available in the Rosetta/Netherlands data is included in Appendix A.

The first set of matrices is built directly from the Rosetta/Netherlands expression ratio data previously described above, that is after the working gene set was selected and expression patterns of selected genes were investigated. Each sample was assigned a group according to the defined constraints, and group members were collected into a single matrix where each row represented one of the genes in the working set, and each column was a sample in the group. A group identification number was added to the data for each sample. The values in this Level 1—Gene matrix represent the transcript abundance as an intensity ratio of sample to reference. A separate matrix is created for each group, resulting in the creation of 6 matrices. Table 4, a Level 1 Matrix of gene expression data, shows the expression values for a subset of receptors and ligands in the EGF family genes for samples in group 4, the ESR1−− samples.

The next level of matrix uses the defined thresholds to identify up- and down-regulated genes by sample. Level 2—Gene Matrix for each group is built directly from the corresponding Level 1—Gene Matrix for that group. Binary values are assigned based on the expression level threshold defined for up-regulated and down-regulated genes. There are separate up- and down-regulated matrices for each group. Like the Level 1—Gene Matrix, each row is a gene and each column is a sample. For up-regulation, a value of 1 is assigned if the gene expression ratio >up-threshold, else 0. For down-regulation, a value of 1 is assigned if ratio is <down-threshold, else 0. For the Rosetta/Netherlands data published in the van't Veer study, the up-threshold selected herein was 0.25 and the down-threshold was −0.25. A final column is added to the matrix which sums the values for each gene across all the samples in the group. Table 5 is an example of this Level 2—Gene Matrix, showing the up-regulation of various the EGF family members across samples in group 4, the ESR1−− group. This matrix was built directly from the matrix illustrated in Table 4. Wherever a value is >0.25 in Table 4, the corresponding value in Table 5 is set to 1. This matrix displays several samples, i.e. Samples 12, 28, 68 and 75 that show up-regulation of several of the ligands in this family. Once again, up- and down-regulation matrices are made for each of the 6 groups defined.

TABLE 5

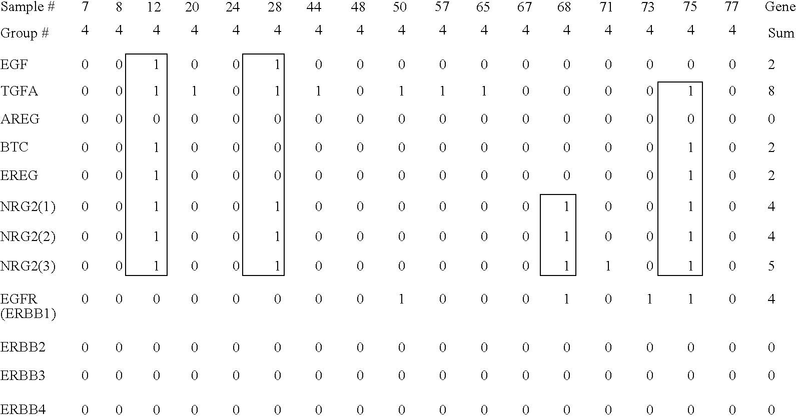

Level 2 Matrix - Gene for Up-Regulation
Excerpt of the members of EGF family of Ligands and Receptors by Sample for Group 4 (ESR1-)

TABLE 6A

| | | Group 1 | Group 2 | Group 3 | Group 4 | Group 5 | Group 6 | Total |
|---|---|---|---|---|---|---|---|---|
| Accession # | Name | N = 16 | N = 15 | N = 17 | N = 17 | N = 13 | N = 20 | N = 98 |
| NM_001963 | EGF | 3 | 1 | 2 | 2 | 2 | 3 | 13 |
| NM_003236 | TGFA | 0 | 0 | 0 | 8 | 1 | 9 | 18 |
| NM_013962 | NRG1 | 0 | 0 | 0 | 5 | 0 | 2 | 7 |
| | (GGF2) | | | | | | | |
| NM_004883 | NRG2(1) | 0 | 1 | 3 | 4 | 0 | 3 | 11 |
| NM_013981 | NRG2(2) | 1 | 0 | 1 | 4 | 1 | 9 | 16 |
| NM_013982 | NRG2(3) | 2 | 0 | 1 | 5 | 1 | 10 | 19 |
| NM_001657 | AREG | 3 | 4 | 3 | 0 | 2 | 0 | 12 |
| NM_005228 | EGFR | 0 | 2 | 1 | 4 | 1 | 0 | 8 |
| | (ERBB1) | | | | | | | |
| NM_004448 | ERBB2 | 0 | 0 | 0 | 0 | 12 | 0 | 12 |
| | (HER2) | | | | | | | |
| NM_001982 | ERBB3 | 3 | 2 | 3 | 0 | 0 | 0 | 8 |
| NM_005235 | ERBB4 | 8 | 4 | 6 | 0 | 1 | 0 | 19 |

Level 3 Matrix - Gene for Up-Regulation EGF Family Ligand and Receptors by Group in Rosetta/Netherlands

TABLE 6B
DOWN REGULATED

| | | Group 1 | Group 2 | Group 3 | Group 4 | Group 5 | Group 6 | Total |
|---|---|---|---|---|---|---|---|---|
| Accession # | Name | N = 16 | N = 15 | N = 17 | N = 17 | N = 13 | N = 20 | N = 98 |
| NM_001963 | EGF | 2 | 3 | 5 | 4 | 8 | 14 | 36 |
| NM_003236 | TGFA | 10 | 8 | 13 | 4 | 9 | 4 | 48 |
| NM_013962 | NRG1 | 1 | 0 | 1 | 0 | 1 | 6 | 9 |
| | (GGF2) | | | | | | | |
| NM_004883 | NRG2(1) | 7 | 5 | 5 | 0 | 8 | 6 | 31 |
| NM_013981 | NRG2(2) | 6 | 2 | 2 | 1 | 9 | 5 | 25 |
| NM_013982 | NRG2(3) | 9 | 4 | 6 | 1 | 7 | 6 | 33 |
| NM_001657 | AREG | 8 | 9 | 9 | 15 | 11 | 18 | 70 |
| NM_005228 | EGFR | 1 | 0 | 4 | 1 | 6 | 8 | 20 |
| | (ERBB1) | | | | | | | |
| NM_004448 | ERBB2 | 16 | 14 | 17 | 17 | 0 | 20 | 84 |
| | (HER2) | | | | | | | |
| NM_001982 | ERBB3 | 1 | 0 | 0 | 11 | 8 | 19 | 39 |
| NM_005235 | ERBB4 | 2 | 3 | 1 | 14 | 11 | 18 | 49 |

Level 3 Matrix - Gene for Down-Regulation EGF Family Ligand and Receptors by Group in Rosetta/Netherlands

The Level 3—Gene Matrix is built from the previously described Level 2—Gene matrices. The data values in this matrix are the counts of up-regulated and down-regulated genes across samples by tumor groups. Again, there are separate matrices for up-regulation and down-regulation. Each column is a tumor group and each row is a gene. For the up-regulation Level 3—Gene Matrix, each value is the number of samples for a given gene in a particular group that are up-regulated. The column for a group in this Level 3—Gene matrix is the Gene Sum column from the Level 2 Matrix that corresponds to the group number. Table 6A is an example of count data for Up-Regulation Level 3—Gene Matrix for the same subset of RTKs and ligands by tumor group previously considered. The values in the column associated with group 4 are taken directly from the Gene Sum column in Table 5. The down-regulation for this set of genes across tumor groups is depicted in Table 6B. Reviewing the matrices, one notes that all of the samples over-expressing ERBB2 or HER2 are in group 5. This is expected because group 5 was defined by overexpression of ERBB2. However, the overexpression of TGFA in groups 4 and group 6 is not the immediate results of constraints imposed on groupings, but a biological phenomenon potentially associated with the lowest level of ESR1 expression.

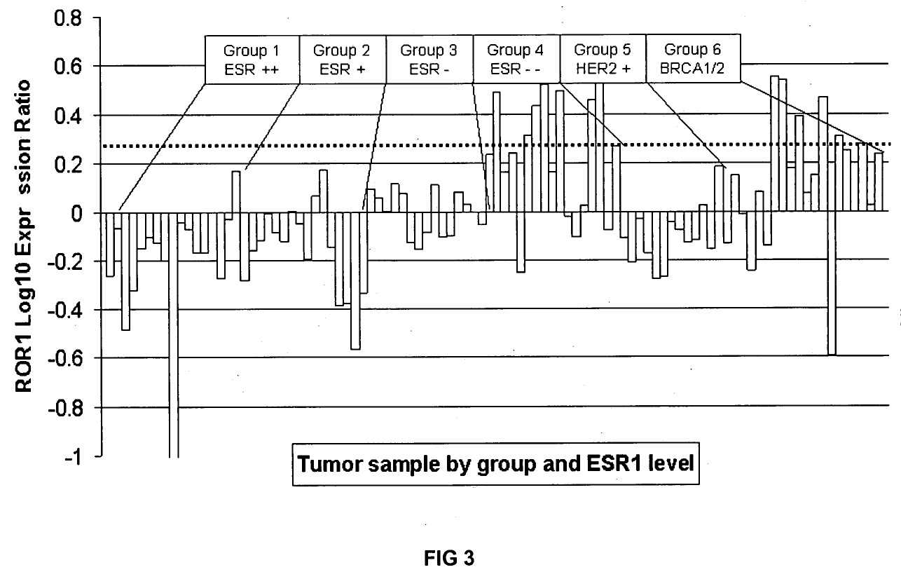

Table 7, the Level 3—Gene Matrix for three related families of receptor tyrosine kinases, shows an interesting finding associated with the pattern of ROR1 overexpression across tumor groups. Musk, NTRKs, and ROR1 & ROR2 comprise three RTK families related by their protein structure. The expression pattern of ROR1 is unique among these receptors, as it is up-regulated specifically in a subset of ESR1−− and BRCA tumors (Groups 4 and 6). To further investigate ROR1 expression, the mRNA expression level of each sample was graphed. (FIG. 3) Samples were organized first by tumor group, and then the level of ESR1 expression within the group. Here it is shown that ROR1 mRNA expression was highest in groups of 4 and 6 (Table 7), that is, tumor groups that were categorized under particular ESR−− and BRCA expression profiles. By including all of the RTKs in the working set, and grouping tumors into subtypes, the pattern of ROR1 expression stands out. It is straightforward to identify ROR1 as a potential target gene, that can be further validated by further experimentation (such as by immunohistochemical and/or molecular analysis studies of samples taken from a subject) as well as a marker or part of a profile that may indicate a candidate for development of various therapeutics (antibody therapies, etc) and/or assays (such as gel assays specific to the newly identified potential target gene) that may indicate a particular prognosis/diagnosis.

TABLE 7
UP REGULATED

| | | Group 1 | Group 2 | Group 3 | Group 4 | Group 5 | Group 6 | |
|---|---|---|---|---|---|---|---|---|
| Accession # | Name | N = 16 | N = 15 | N = 17 | N = 17 | N = 13 | N = 20 | Total N = 98 |
| NM_005592 | MUSK | 1 | 0 | 3 | 0 | 0 | 1 | 5 |
| NM_002507 | NGFR | 1 | 0 | 0 | 1 | 0 | 0 | 2 |
| NM_002529 | NTRK1 | 0 | 0 | 3 | 1 | 0 | 1 | 5 |
| | (TRKA) | | | | | | | |
| NM_006180 | NTRK2 | 4 | 1 | 2 | 5 | 0 | 1 | 13 |
| | (TRKB) | | | | | | | |
| NM_002530 | NTRK3 | 6 | 4 | 5 | 4 | 0 | 0 | 19 |
| | (TRKC) | | | | | | | |
| NM_005012 | ROR1 | 0 | 0 | 0 | 8 | 0 | 7 | 15 |
| NM_004560 | ROR2 | 0 | 2 | 1 | 1 | 2 | 1 | 7 |

Level 3 Matrix - Gene for Up-Regulation Related Receptor Tyrosine Kinase by Group in Rosetta/Netherlands Data

Tables 8 and 9 demonstrate another level of analysis based on the expression of members of gene sets, rather than individual genes. Table 8 is an up-regulation Level 3—Gene Set Matrix for group 6, the BRCA group. Each column is a sample assigned to group 6, and each row represents a set of genes representing the receptors of an RTK family that bind to the same ligand or ligands. Each value is the count of the number of up-regulated receptors for that gene set and sample. This matrix is built by summing the appropriate cells in the Level 2—Gene matrix for the appropriate group. For example, the row labeled “FGFs” in Table 8, sums up the values for the six genes associated with the fibroblast growth factor receptor family. (See Appendix A). The Sum column adds up all of the values for that row or gene set. Table 9 is the same type of matrix for Ligand expression by sample in Group 6.

TABLE 8

| | Sample # | | | | | | | | | | | | | | | | | | | | |
|---|---|---|---|---|---|---|---|---|---|---|---|---|---|---|---|---|---|---|---|---|---|
| | 80 | 81 | 83 | 84 | 85 | 86 | 87 | 88 | 89 | 90 | 91 | 92 | 93 | 95 | 96 | 97 | 98 | 100 | 94 | 99 | |
| | Group # | | | | | | | | | | | | | | | | | | | | |
| | 6 | 6 | 6 | 6 | 6 | 6 | 6 | 6 | 6 | 6 | 6 | 6 | 6 | 6 | 6 | 6 | 6 | 6 | 6 | 6 | Sum |
| EGFR | 0 | 0 | 0 | 0 | 0 | 0 | 0 | 0 | 0 | 0 | 0 | 0 | 0 | 0 | 0 | 0 | 0 | 0 | 0 | 0 | 0 |
| ERBB2, ERBB3, ERBB4 | 0 | 0 | 0 | 0 | 0 | 0 | 0 | 0 | 0 | 0 | 0 | 0 | 0 | 0 | 0 | 0 | 0 | 0 | 0 | 0 | 0 |
| IGF1R, INSR, CROS, INSRR | 0 | 0 | 0 | 0 | 0 | 0 | 0 | 0 | 0 | 0 | 0 | 0 | 0 | 0 | 1 | 0 | 0 | 0 | 0 | 0 | 1 |
| EPHAs | 0 | 0 | 1 | 0 | 1 | 0 | 1 | 0 | 1 | 1 | 0 | 1 | 1 | 0 | 0 | 0 | 0 | 0 | 0 | 0 | 7 |
| EPHBs | 2 | 0 | 0 | 2 | 0 | 1 | 2 | 4 | 1 | 1 | 0 | 2 | 3 | 0 | 0 | 0 | 0 | 3 | 0 | 0 | 21 |
| AXL, TYRO3, MERTK | 0 | 0 | 0 | 1 | 0 | 0 | 0 | 0 | 0 | 0 | 0 | 0 | 0 | 0 | 0 | 0 | 0 | 0 | 0 | 0 | 1 |
| TIE, TEK | 0 | 0 | 0 | 0 | 0 | 0 | 0 | 0 | 0 | 0 | 0 | 0 | 0 | 0 | 0 | 0 | 0 | 0 | 0 | 0 | 0 |
| PDGFs | 0 | 0 | 0 | 0 | 0 | 0 | 1 | 0 | 0 | 0 | 0 | 0 | 0 | 2 | 1 | 0 | 0 | 0 | 0 | 0 | 4 |
| KIT, CSF1R, FLT3 | 0 | 0 | 1 | 0 | 0 | 0 | 1 | 0 | 1 | 0 | 0 | 0 | 0 | 1 | 0 | 2 | 1 | 0 | 0 | 1 | 8 |
| FLT1, KDR, FLT4 | 0 | 0 | 0 | 0 | 0 | 0 | 0 | 0 | 0 | 0 | 0 | 1 | 0 | 0 | 0 | 0 | 0 | 0 | 0 | 0 | 1 |
| FGFRs | 0 | 0 | 0 | 1 | 0 | 0 | 0 | 0 | 1 | 0 | 0 | 0 | 0 | 0 | 0 | 0 | 0 | 0 | 0 | 0 | 2 |
| PTK7 | 0 | 0 | 0 | 0 | 0 | 0 | 1 | 1 | 0 | 0 | 0 | 0 | 1 | 0 | 0 | 0 | 0 | 0 | 0 | 0 | 3 |
| MUSK | 0 | 0 | 0 | 0 | 0 | 0 | 0 | 0 | 0 | 0 | 0 | 0 | 0 | 0 | 1 | 0 | 0 | 0 | 0 | 0 | 1 |
| NGFR, NTRKs | 0 | 0 | 1 | 0 | 0 | 0 | 0 | 0 | 0 | 0 | 0 | 0 | 1 | 0 | 0 | 0 | 0 | 0 | 0 | 0 | 2 |
| ROR1, ROR2 | 1 | 0 | 0 | 0 | 0 | 0 | 1 | 1 | 0 | 0 | 1 | 1 | 1 | 0 | 0 | 0 | 1 | 1 | 0 | 0 | 8 |
| RYK | 0 | 0 | 0 | 0 | 0 | 1 | 0 | 0 | 0 | 0 | 0 | 0 | 1 | 0 | 0 | 0 | 0 | 0 | 0 | 0 | 2 |
| DDR1, DDR2 | 0 | 0 | 0 | 0 | 0 | 0 | 0 | 0 | 0 | 0 | 0 | 1 | 0 | 0 | 0 | 0 | 0 | 1 | 0 | 1 | 3 |
| RET | 0 | 0 | 0 | 0 | 0 | 0 | 0 | 0 | 0 | 0 | 0 | 0 | 0 | 0 | 0 | 0 | 0 | 0 | 0 | 0 | 0 |
| MET, MST1R | 0 | 0 | 0 | 0 | 0 | 0 | 0 | 0 | 0 | 0 | 0 | 0 | 0 | 0 | 0 | 0 | 0 | 0 | 0 | 0 | 0 |

Level 3 Matrix - Gene Set for Up-Regulation in Group 6 (BRCA)Receptor Tyrosine Kinase expression by family across samples

TABLE 9

| | Sample # | | | | | | | | | | | | | | | | | | | | |
|---|---|---|---|---|---|---|---|---|---|---|---|---|---|---|---|---|---|---|---|---|---|
| | 80 | 81 | 83 | 84 | 85 | 86 | 87 | 88 | 89 | 90 | 91 | 92 | 93 | 95 | 96 | 97 | 98 | 100 | 94 | 99 | |
| | Group # | | | | | | | | | | | | | | | | | | | | |
| | 6 | 6 | 6 | 6 | 6 | 6 | 6 | 6 | 6 | 6 | 6 | 6 | 6 | 6 | 6 | 6 | 6 | 6 | 6 | 6 | Sum |
| NRGs | 0 | 3 | 2 | 4 | 0 | 0 | 1 | 0 | 3 | 2 | 2 | 1 | 2 | 9 | 0 | 2 | 0 | 0 | 0 | 2 | 33 |
| EGFs | 0 | 0 | 0 | 3 | 2 | 1 | 1 | 1 | 2 | 1 | 1 | 0 | 0 | 0 | 0 | 1 | 1 | 1 | 1 | 1 | 17 |
| IGFs | 0 | 0 | 0 | 0 | 0 | 0 | 0 | 0 | 1 | 1 | 0 | 0 | 0 | 1 | 0 | 0 | 0 | 0 | 0 | 0 | 3 |
| INSLs | 1 | 0 | 0 | 2 | 0 | 0 | 1 | 0 | 0 | 0 | 0 | 0 | 1 | 0 | 0 | 0 | 0 | 0 | 1 | 0 | 6 |
| PTN | 0 | 0 | 0 | 0 | 0 | 0 | 0 | 0 | 0 | 0 | 0 | 0 | 0 | 0 | 0 | 0 | 0 | 0 | 0 | 0 | 0 |
| EFNAs | 0 | 0 | 0 | 0 | 0 | 0 | 0 | 0 | 0 | 0 | 0 | 0 | 0 | 0 | 0 | 0 | 0 | 1 | 0 | 0 | 1 |
| EFNBs | 0 | 0 | 1 | 1 | 0 | 0 | 0 | 0 | 1 | 0 | 0 | 0 | 1 | 0 | 0 | 0 | 0 | 0 | 0 | 0 | 4 |
| GAS6&PROS1 | 0 | 0 | 0 | 0 | 0 | 0 | 0 | 0 | 0 | 0 | 0 | 1 | 0 | 1 | 0 | 0 | 1 | 1 | 0 | 0 | 4 |
| ANGPTs | 1 | 0 | 1 | 0 | 0 | 0 | 0 | 0 | 0 | 0 | 0 | 1 | 0 | 0 | 0 | 0 | 0 | 1 | 0 | 0 | 4 |
| PDGFs | 1 | 0 | 0 | 0 | 0 | 0 | 2 | 0 | 0 | 0 | 0 | 0 | 0 | 1 | 1 | 0 | 0 | 0 | 0 | 0 | 5 |
| SCF, KITLG, CSF1, FLT3LG | 0 | 0 | 0 | 0 | 0 | 0 | 0 | 0 | 1 | 1 | 0 | 0 | 0 | 0 | 0 | 0 | 0 | 0 | 0 | 0 | 2 |
| VEGFs | 1 | 0 | 1 | 0 | 0 | 0 | 1 | 1 | 0 | 0 | 1 | 2 | 1 | 1 | 1 | 1 | 0 | 0 | 0 | 1 | 12 |
| FGFs | 0 | 3 | 1 | 1 | 0 | 0 | 1 | 0 | 2 | 3 | 1 | 2 | 1 | 3 | 3 | 1 | 2 | 0 | 1 | 0 | 25 |
| NGFB, BDNF, NTF3 | 0 | 0 | 1 | 0 | 0 | 0 | 1 | 0 | 0 | 0 | 0 | 0 | 0 | 0 | 0 | 0 | 0 | 0 | 0 | 0 | 2 |
| GDNF&ARTN | 0 | 0 | 0 | 0 | 0 | 0 | 0 | 0 | 0 | 0 | 0 | 0 | 0 | 0 | 0 | 0 | 0 | 0 | 0 | 0 | 0 |
| HGF&MST1 | 0 | 0 | 1 | 1 | 0 | 0 | 0 | 0 | 0 | 1 | 0 | 0 | 0 | 0 | 0 | 0 | 0 | 0 | 1 | 0 | 4 |

Level 3 Matrix - Gene Set for Up-Regulation in Group 6 (BRCA) Ligand expression by family across samples

Table 10 combines the information included in Tables 8 and 9. It is an example of a Level 2 Matrix for gene set unions, here an RTK gene set and Ligand gene set into RTK/Ligand pair by sample in Group 6 (BRCA). Each column is a sample assigned to the BRCA group. Each row represents the union of a ligand gene set and the associated receptor gene set. The data values are binary; a value if 1 is assigned if both the ligand and receptor value are greater than one in the appropriate gene set matrix. If either the receptor or the ligands for a gene family is unknown, the data values in the row are left blank. Table 10 shows the up-regulation Level 2 Matrix for the union of RTK and ligand gene sets for group 6. Looking at this matrix, it is clear that few samples show up-regulation for both ligand and receptors for any of the RTK families. Only the Ephrin B subfamily, PDGF family and FGF family are up-regulated in more than one sample in this group.

TABLE 10

| | | Sample # | | | | | | | | | | | | | | | | | | | | RTK |
|---|---|---|---|---|---|---|---|---|---|---|---|---|---|---|---|---|---|---|---|---|---|---|
| | | 80 | 81 | 83 | 84 | 85 | 86 | 87 | 88 | 89 | 90 | 91 | 92 | 93 | 95 | 96 | 97 | 98 | 100 | 94 | 99 | Group |
| Ligands | Receptors | 6 | 6 | 6 | 6 | 6 | 6 | 6 | 6 | 6 | 6 | 6 | 6 | 6 | 6 | 6 | 6 | 6 | 6 | 6 | 6 | Sum |
| NRGs | ERBB2, ERBB3, | 0 | 0 | 0 | 0 | 0 | 0 | 0 | 0 | 0 | 0 | 0 | 0 | 0 | 0 | 0 | 0 | 0 | 0 | 0 | 0 | 0 |
| | ERBB4 | | | | | | | | | | | | | | | | | | | | | |
| EGFs | EGFR | 0 | 0 | 0 | 0 | 0 | 0 | 0 | 0 | 0 | 0 | 0 | 0 | 0 | 0 | 0 | 0 | 0 | 0 | 0 | 0 | 0 |
| IGFs | IGF1R, INSR, | 0 | 0 | 0 | 0 | 0 | 0 | 0 | 0 | 0 | 0 | 0 | 0 | 0 | 0 | 0 | 0 | 0 | 0 | 0 | 0 | 0 |
| | CROS, INSRR | | | | | | | | | | | | | | | | | | | | | |
| INSLs | Unknown | | | | | | | | | | | | | | | | | | | | | 0 |
| PTN | Unknown | | | | | | | | | | | | | | | | | | | | | 0 |
| EFNAs | EPHAs | 0 | 0 | 0 | 0 | 0 | 0 | 0 | 0 | 0 | 0 | 0 | 0 | 0 | 0 | 0 | 0 | 0 | 0 | 0 | 0 | 0 |
| EFNBs | EPHBs | 0 | 0 | 0 | 1 | 0 | 0 | 0 | 0 | 1 | 0 | 0 | 0 | 1 | 0 | 0 | 0 | 0 | 0 | 0 | 0 | 3 |
| GAS6&PROS1 | AXL, TYRO3, MERTK | 0 | 0 | 0 | 0 | 0 | 0 | 0 | 0 | 0 | 0 | 0 | 0 | 0 | 0 | 0 | 0 | 0 | 0 | 0 | 0 | 0 |
| ANGPTs | TIE, TEK | 0 | 0 | 0 | 0 | 0 | 0 | 0 | 0 | 0 | 0 | 0 | 0 | 0 | 0 | 0 | 0 | 0 | 0 | 0 | 0 | 0 |
| PDGFs | PDGFs | 0 | 0 | 0 | 0 | 0 | 0 | 1 | 0 | 0 | 0 | 0 | 0 | 0 | 1 | 1 | 0 | 0 | 0 | 0 | 0 | 3 |
| SCF, KITLG, | KIT, CSF1R, FLT3 | 0 | 0 | 0 | 0 | 0 | 0 | 0 | 0 | 1 | 0 | 0 | 0 | 0 | 0 | 0 | 0 | 0 | 0 | 0 | 0 | 1 |
| CSF1, | | | | | | | | | | | | | | | | | | | | | | |
| FLT3LG | | | | | | | | | | | | | | | | | | | | | | |
| VEGFs | FLT1, KDR, FLT4 | 0 | 0 | 0 | 0 | 0 | 0 | 0 | 0 | 0 | 0 | 0 | 1 | 0 | 0 | 0 | 0 | 0 | 0 | 0 | 0 | 1 |
| FGFs | FGFRs | 0 | 0 | 0 | 1 | 0 | 0 | 0 | 0 | 1 | 0 | 0 | 0 | 0 | 0 | 0 | 0 | 0 | 0 | 0 | 0 | 2 |
| Unknown | PTK7 | | | | | | | | | | | | | | | | | | | | | 0 |
| Unknown | MUSK | | | | | | | | | | | | | | | | | | | | | 0 |
| NGFB, BDNF, | NGFR, NTRKs | 0 | 0 | 1 | 0 | 0 | 0 | 0 | 0 | 0 | 0 | 0 | 0 | 0 | 0 | 0 | 0 | 0 | 0 | 0 | 0 | 1 |
| NTF3 | | | | | | | | | | | | | | | | | | | | | | |
| Unknown | ROR1, ROR2 | | | | | | | | | | | | | | | | | | | | | 0 |
| Unknown | RYK | | | | | | | | | | | | | | | | | | | | | 0 |
| Unknown | DDR1, DDR2 | | | | | | | | | | | | | | | | | | | | | 0 |
| GDNF&ARTN | RET | 0 | 0 | 0 | 0 | 0 | 0 | 0 | 0 | 0 | 0 | 0 | 0 | 0 | 0 | 0 | 0 | 0 | 0 | 0 | 0 | 0 |
| HGF&MST1 | MET, MST1R | 0 | 0 | 0 | 0 | 0 | 0 | 0 | 0 | 0 | 0 | 0 | 0 | 0 | 0 | 0 | 0 | 0 | 0 | 0 | 0 | 0 |
| | Sample Sum | 0 | 0 | 1 | 2 | 0 | 0 | 1 | 0 | 3 | 0 | 0 | 1 | 1 | 1 | 1 | 0 | 0 | 0 | 0 | 0 | 11 |

Level 2 Matrix—Gene Set Union for Up-Regulation in Group 6 (BRCA)
Receptor/Ligand co expression by family across samples

References to various works have been cited herein and all are incorporated by reference in their entirety as if each work had been incorporated by reference individually.

Although the present invention has been described in connection with the preferred form of practicing it, those of ordinary skill in the art will understand that many modifications can be made thereto without departing from the spirit of the present invention. Accordingly, it is not intended that the scope of the invention in any way be limited by the above description.

References

Perou et al. (2000) “Molecular Portraits of human breast tumors”. Nature (406) 747-752.

Tibshirani et al. (2002) “Diagnosis of multiple cancer types by shrunken centroids of gene expression.” PNAS (99) 6567-6572.

Tukey, John W. (1977) Exploratory Data Analysis. Massachusetts:Addison Wesley.

Sorlie et al. (2001) “Gene expression patterns of breast carcinomas distinguish tumor subclasses with clinical implications”. PNAS (98) 10869-10874.

van t Veer et al. (2002) “Gene expression profiling predicts clinical outcome of breast cancer”. Nature (415) 530-536.

APPENDIX A

| | Accession # | Gene Name | Description | R/L | Group |
|---|---|---|---|---|---|
| 1 | NM_001963 | EGF | epidermal growth factor (beta-urogastrone) | L | 1 |
| 2 | NM_003236 | TGFA | transforming growth factor, alpha | L | 1 |
| 3 | NM_004495 | NRG1 (HRG- | neuregulin 1 | L | 1 |
| | | Gamma) | | | |
| 4 | NM_013956 | NRG1 (HRG-Beta1) | neuregulin 1 | L | 1 |
| 5 | NM_013957 | NRG1 (HRG-Beta2) | neuregulin 1 | L | 1 |
| 6 | NM_013958 | NRG1 (HRG-Beta3) | neuregulin 1 | L | 1 |
| 7 | NM_013960 | NRG1 (ndf43) | neuregulin 1 | L | 1 |
| 8 | NM_013961 | NRG1 (GGF) | neuregulin 1 | L | 1 |
| 9 | NM_013962 | NRG1 (GGF2) | neuregulin 1 | L | 1 |
| 10 | NM_004883 | NRG2(1) | neuregulin 2 | L | 1 |
| 11 | NM_013981 | NRG2(2) | neuregulin 2 | L | 1 |
| 12 | NM_013982 | NRG2(3) | neuregulin 2 | L | 1 |
| 13 | NM_013984 | NRG2(5) | neuregulin 2 | L | 1 |
| 14 | NM_013985 | NRG2(6) | neuregulin 2 | L | 1 |
| 15 | NM_001945 | DTR (HBEGF) | diphtheria toxin receptor (heparin-binding epidermal growth factor-like | L | 1 |
| | | | growth factor) | | |
| 16 | NM_001657 | AREG | amphiregulin (schwannoma-derived growth factor) | L | 1 |
| 18 | NM_001729 | BTC | betacellulin | L | 1 |
| 19 | NM_001432 | EREG | epiregulin | | 1 |
| 20 | NM_005228 | EGFR (ERBB1) | epidermal growth factor receptor (avian erythroblastic leukemia viral | R | 1 |
| | | | (v-erb-b) oncogene homolog) | | |
| 21 | NM_004448 | ERBB2 | v-erb-b2 avian erythroblastic leukemia viral oncogene homolog 2 | R | 1 |
| | | | (neuro/glioblastoma derived oncogene homolog) | | |
| 22 | NM_001982 | ERBB3 | v-erb-b2 avian erythroblastic leukemia viral oncogene homolog 3 | R | 1 |
| 23 | NM_005235 | ERBB4 | v-erb-a avian erythroblastic leukemia viral oncogene homolog-like 4 | R | 1 |
| 24 | NM_000207 | INS | insulin | L | 1 |
| 25 | X57025 | IGF1 | insulin-like growth factor 1 (somatomedia C) | L | 2 |
| 26 | NM_000612 | IGF2 | insulin-like growth factor 2 (somatomedin A) | L | 2 |
| 27 | NM_012421 | RLF (INSL3) ? | rearranged L-myc fusion sequence | L | 2 |
| 28 | NM_002195 | INSL4 | insulin-like 4 (placenta) | L | 2 |
| 29 | NM_005478 | INSL5 | insulin-like 5 | L | 2 |
| 30 | NM_007179 | INSL6 | insulin-like 6 | L | 2 |
| 31 | NM_006911 | RLN1 (REL1) | relaxin 1 (H1) | L | 2 |
| 32 | NM_005059 | RLN2 (REL2) | relaxin 2 (H2) | L | 2 |
| 33 | J05046 | INSRR (IRR) | insulin receptor-related receptor | R | 2 |
| 34 | NM_000208 | INSR (IR) | insulin receptor | R | 2 |
| 35 | NM_000875 | IGF1R | insulin-like growth factor 1 receptor | R | 2 |
| 36 | NM_000876 | IGF2R | insulin-like growth factor 2 receptor | R | 2 |
| 37 | NM_002944 | ROS1 (C-ROS) | v-ros avian UR2 sarcoma virus oncogene homolog 1 | R | 2 |
| 38 | NM_002825 | PTN | pleiotrophin (heparin binding growth factor 8, neurite growth- | L | 3 |
| | | | promoting factor 1) | | |
| 39 | NM_004304 | ALK | anaplastic lymphoma kinase (Ki-1) | R | 3 |
| 40 | NM_002344 | LTK | leukocyte tyrosine kinase | R | 3 |
| 41 | NM_004428 | EFNA1 | ephrin-A1 | L | 4 |
| 42 | NM_004952 | EFNA3 | ephrin-A3 | L | 4 |
| 43 | NM_005227 | EFNA4 | ephrin-A4 | L | 4 |
| 44 | NM_001962 | EFNA5 | ephrin-A5 | L | 4 |
| 45 | NM_004429 | EFNB1 | ephrin-B1 | L | 4 |
| 46 | NM_004093 | EFNB2 | ephrin-B2 | L | 4 |
| 47 | NM_001406 | EFNB3 | ephrin-B3 | L | 4 |
| 48 | NM_005232 | EPHA1 | EphA1 | R | 4 |
| 49 | NM_004431 | EPHA2 | EphA2 | R | 4 |
| 50 | NM_005233 | EPHA3 | EphA3 | R | 4 |
| 51 | NM_004438 | EPHA4 | EphA4 | R | 4 |
| 52 | X95425 | EPHA5 | EphA5 | R | 4 |
| 53 | NM_004440 | EPHA7 | EphA7 | R | 4 |
| 54 | NM_020526 | EPHA8 | Homo sapiens EphA8 (EPHA8), mRNA. | R | 4 |
| 55 | AB040892 | EPHA8 | EphA8 | R | 4 |
| 56 | NM_004441 | EPHB1 | EphB1 | R | 4 |
| 57 | Contig49445_RC | EPHB2 | ESTs | R | 4 |
| 58 | AF025304 | EPHB2 | EphB2 | R | 4 |
| 59 | NM_004443 | EPHB3 | EphB3 | R | 4 |
| 60 | NM_004444 | EPHB4 | EphB4 | R | 4 |
| 61 | NM_004445 | EPHB6 | EphB6 | R | 4 |
| 62 | NM_000820 | GAS6 | growth arrest-specific 6 | L | 5 |
| 63 | NM_000313 | PROS1 | protein S (alpha) | R | 5 |
| 64 | NM_001699 | AXL | AXL receptor tyrosine kinase | R | 5 |
| 66 | NM_006293 | TYRO3 | TYRO3 protein tyrosine kinase | R | 5 |
| 67 | NM_006343 | MERTK (c-Mer) | c-mer proto-oncogene tyrosine kinase | R | 5 |
| 68 | NM_001146 | ANGPT1 | angiopoietin 1 | L | 6 |
| 69 | NM_001147 | ANGPT2 | angiopoietin 2 | L | 6 |
| 70 | NM_005424 | TIE (TIE1) | tyrosine kinase with immunoglobulin and epidermal growth factor | R | 6 |
| | | | homology domains | | |
| 71 | NM_000459 | TEK (TIE2) | TEK tyrosine kinase, endothelial (venous malformations, multiple | R | 6 |
| | | | cutaneous and mucosal) | | |
| 72 | NM_002607 | PDGFA | platelet-derived growth factor alpha polypeptide | L | 7 |
| 73 | NM_002608 | PDGFB | platelet-derived growth factor beta polypeptide (simian sarcoma viral | L | 7 |
| | | | (v-sis) oncogene homolog) | | |
| 74 | NM_016205 | PDGFC | Homo sapiens platelet derived growth factor C (PDGFC), mRNA. | L | 7 |
| 75 | AF091434 | PDGFC | platelet derived growth factor C | L | 7 |
| 76 | S80491 | stem cell factor, | Stem cell factor {alternatively spliced} [human, preimplantation | L | 7 |
| | | SCF | embryos, blastocysts, mRNA Partial, 180 nt] | | |
| 77 | NM_003994 | KITLG | KIT ligand | L | 7 |
| 78 | NM_000899 | KITLG | KIT ligand | L | 7 |
| 79 | NM_000757 | CSF1 | colony stimulating factor 1 (macrophage) | L | 7 |
| 80 | NM_001459 | FLT3LG | fms-related tyrosine kinase 3 ligand | L | 7 |
| 81 | X76079 | PDGFRA | Human platelet-derived growth factor alpha-receptor (PDGFRA) | R | 7 |
| | | | mRNA, exons 13-16 | | |
| 82 | NM_006206 | PDGFRA | platelet-derived growth factor receptor, alpha polypeptide | R | 7 |
| 83 | NM_002609 | PDGFRB | platelet-derived growth factor receptor, beta polypeptide | R | 7 |
| 84 | NM_000222 | KIT (C-KIT) | v-kit Hardy-Zuckerman 4 feline sarcoma viral oncogene homolog | R | 7 |
| 85 | NM_005211 | CSF1R | colony stimulating factor 1 receptor, formerly McDonough feline | R | 7 |
| | | | sarcoma viral (v-fms) oncogene homolog | | |
| 86 | NM_004119 | FLT3 | fms-related tyrosine kinase 3 | R | 7 |
| 87 | NM_003376 | VEGF | vascular endothelial growth factor | L | 8 |
| 88 | NM_003377 | VEGFB | vascular endothelial growth factor B | L | 8 |
| 89 | NM_005429 | VEGFC | vascular endothelial growth factor C | L | 8 |
| 90 | NM_004469 | FIGF (VEGFD) | c-fos induced growth factor (vascular endothelial growth factor D) | L | 8 |
| 91 | NM_002632 | PGF (PLGF) | placental growth factor, vascular endothelial growth factor-related | L | 8 |
| | | | protein | | |
| 92 | NM_002019 | FLT1 (VGFR1) | fms-related tyrosine kinase 1 (vascular endothelial growth | R | 8 |
| | | | factor/vascular permeability factor receptor) | | |
| 93 | AF035121 | KDR (VGFR2) | kinase insert domain receptor (a type III receptor tyrosine kinase) | R | 8 |
| 94 | NM_002020 | FLT4 (VGFR3) | fms-related tyrosine kinase 4 | R | 8 |
| 95 | NM_000800 | FGF1 | fibroblast growth factor 1 (acidic) | L | 9 |
| 96 | NM_002006 | FGF2 | fibroblast growth factor 2 (basic) | L | 9 |
| 97 | NM_005247 | FGF3 | fibroblast growth factor 3 (murine mammary tumor virus integration | L | 9 |
| | | | site (v-int-2) oncogene homolog) | | |
| 98 | NM_002007 | FGF4 | fibroblast growth factor 4 (heparin secretory transforming protein 1, | L | 9 |
| | | | Kaposi sarcoma oncogene) | | |
| 99 | NM_004464 | FGF5 | fibroblast growth factor 5 | L | 9 |
| 100 | NM_020996 | FGF6 | Homo sapiens fibroblast growth factor 6 (FGF6), mRNA. | L | 9 |
| 101 | X63454 | FGF6 | fibroblast growth factor 6 | L | 9 |
| 102 | NM_002009 | FGF7 | fibroblast growth factor 7 (keratinocyte growth factor) | L | 9 |
| 103 | NM_006119 | FGF8 | fibroblast growth factor 8 (androgen-induced) | L | 9 |
| 104 | NM_002010 | FGF9 | fibroblast growth factor 9 (glia-activating factor) | L | 9 |
| 105 | NM_004465 | FGF10 | fibroblast growth factor 10 | L | 9 |
| 106 | Contig49632_RC | FGF11 | fibroblast growth factor 11 | L | 9 |
| 107 | NM_004112 | FGF11 | fibroblast growth factor 11 | L | 9 |
| 108 | NM_004113 | FGF12B | fibroblast growth factor 12B | L | 9 |
| 109 | U66197 | FGF12 | fibroblast growth factor 12 | L | 9 |
| 110 | NM_004114 | FGF13 | fibroblast growth factor 13 | L | 9 |
| 111 | NM_004115 | FGF14 | fibroblast growth factor 14 | L | 9 |
| 112 | NM_003868 | FGF16 | fibroblast growth factor 16 | L | 9 |
| 113 | NM_003867 | FGF17 | fibroblast growth factor 17 | L | 9 |
| 114 | NM_003862 | FGF18 | fibroblast growth factor 18 | L | 9 |
| 115 | NM_005117 | FGF19 | fibroblast growth factor 19 | L | 9 |
| 116 | NM_019851 | FGF20 | fibroblast growth factor 20 | L | 9 |
| 117 | NM_019113 | FGF21 | fibroblast growth factor 21 | L | 9 |
| 118 | NM_020638 | FGF23 | Homo sapiens fibroblast growth factor 23 (FGF23), mRNA. | L | 9 |
| 119 | X66945 | FGFR1 | fibroblast growth factor receptor 1 (fms-related tyrosine kinase 2, | R | 9 |
| | | | Pfeiffer syndrome) | | |
| 120 | NM_015850 | FGFR1 | fibroblast growth factor receptor 1 (fms-related tyrosine kinase 2, | R | 9 |
| | | | Pfeiffer syndrome) | | |
| 121 | NM_000141 | FGFR2 | fibroblast growth factor receptor 2 (bacteria-expressed kinase, | R | 9 |
| | | | keratinocyte growth factor receptor, craniofacial dysostosis 1, | | |
| | | | Crouzon syndrome, Pfeiffer syndrome, Jackson-Weiss syndrome) | | |
| 122 | NM_000142 | FGFR3 | fibroblast growth factor receptor 3 (achondroplasia, thanatophoric | R | 9 |
| | | | dwarfism) | | |
| 123 | AF202063 | FGFR4 | fibroblast growth factor receptor 4 | R | 9 |
| 124 | NM_002011 | FGFR4 | fibroblast growth factor receptor 4 | R | 9 |
| 125 | NM_002821 | PTK7 | PTK7 protein tyrosine kinase 7 | R | 10 |
| 126 | AF016903 | AGRN | Homo sapiens agrin precursor mRNA, partial cds | L | 11 |
| 127 | NM_005592 | MUSK | muscle, skeletal, receptor tyrosine kinase | R | 11 |
| 128 | NM_002506 | NGFB (NGF) | nerve growth factor, beta polypeptide | L | 12 |
| 129 | NM_001709 | BDNF | brain-derived neurotrophic factor | L | 12 |
| 130 | NM_002527 | NTF3 (NT3) | neurotrophin 3 | L | 12 |
| 131 | Contig873_RC | NT5 (NT4?) | ESTs ??? GeneCard has NTF4 as a synonym for neurotrophin (4/5); | L | 12 |
| | | | the canonical name assigned to the gene is NTF5. None of the | | |
| | | | following are in the data: NTF4, NTF5, NT-4/5. The closest symbol I | | |
| | | | could find was NT5, and there is no description for this ge | | |
| 132 | NM_002507 | NGFR | nerve growth factor receptor (TNFR superfamily, member 16) | R | 12 |
| 133 | NM_002529 | NTRK1 (TRKA) | neurotrophic tyrosine kinase, receptor, type 1 | R | 12 |
| 134 | NM_006180 | NTRK2 (TRKB) | neurotrophic tyrosine kinase, receptor, type 2 | R | 12 |
| 135 | NM_002530 | NTRK3 (TRKC) | neurotrophic tyrosine kinase, receptor, type 3 | R | 12 |
| 136 | NM_005012 | ROR1 | receptor tyrosine kinase-like orphan receptor 1 | R | 13 |
| 137 | NM_004560 | ROR2 | receptor tyrosine kinase-like orphan receptor 2 | R | 13 |
| 138 | S59184 | RYK | RYK receptor-like tyrosine kinase | R | 14 |
| 139 | NM_001954 | DDR1 | discoidin domain receptor family, member 1 | R | 15 |
| 140 | NM_013994 | DDR1 | discoidin domain receptor family, member 1 | R | 15 |
| 141 | NM_006182 | DDR2 | discoidin domain receptor family, member 2 | R | 15 |
| 142 | NM_000514 | GDNF | glial cell derived neurotrophic factor | L | 16 |
| 143 | NM_003976 | ARTN (Artemin) | artemin | L | 16 |
| 144 | NM_000323 | RET | Homo sapiens ret proto-oncogene (multiple endocrine neoplasia | R | 16 |
| | | | MEN2A, MEN2B and medullary thyroid carcinoma 1, Hirschsprung | | |
| | | | disease) (RET), transcript variant 1, mRNA. | | |
| 145 | X16323 | HGF | hepatocyte growth factor (hepapoietin A; scatter factor) | L | 17 |
| 146 | NM_020998 | MST1 (MSP) | Homo sapiens macrophage stimulating 1 (hepatocyte growth factor- | L | 17 |
| | | | like) (MST1), mRNA. | | |
| 147 | L11924 | MST1 (MSP) | macrophage stimulating 1 (hepatocyte growth factor-like) | L | 17 |
| 148 | NM_000245 | MET | met proto-oncogene (hepatocyte growth factor receptor) | R | 17 |
| 149 | NM_002447 | MST1R (RON) | macrophage stimulating 1 receptor (c-met-related tyrosine kinase) | R | 17 |

1. A constraint-based method for identifying a genomic target of interest from a gene expression profiles, comprising:
obtaining tissue sample expression data sets;
selecting a working gene-expressionset, said gene-expression set having a plurality of members
defining subgroups of said tissue samples of said expression data sets, wherein said subgroups definition is a constrained definition; and
analyzing co-expression of said members gene set across said subgroups and identifying potential gene targets.

2 The method of claim 1, wherein said tissue sample expression data sets are comprised of expressions data sets from tumor samples.

3 The method of claim 1, wherein said tissue sample expression data sets are comprised of expression data sets from tissue from a mammal.

4 The method of claim 3, wherein said tissue sample expression data sets are comprised of expression data sets attained tissue from a least one of a human, mouse, primate, canine, pig, rat, and feline.

5 The method of claim 1, wherein said tissue sample expression data sets are comprised of expression data sets based upon tissue from and embryo.

6 The method of claim 2, wherein said tumor are breast cancer tumors.

7 The method of claim 2 wherein said tumors are cancer tumor of the digestive tract.

8 The method of claim 1, wherein said working gene expression set comprises at least one receptor tyrosine kinase.

9 The method of claim 1, wherein said working gene expression set comprises a receptor and a ligand.

10 The method of claim 1, further comprising a step of selecting known prognostic markers that are correlative with prognostic outcomes.

11. The method of claim 1, further comprising a step of binning said working gene-expression set.

12. A constraint-based method for analysis of gene expression profiles, comprising:
selecting a working gene set;
investigating expression patterns of said working gene set in a set of tissue samples;
defining cutting values to define categories of gene expression levels;
selecting constraints in order to bin said tissue samples into groups according to gene expression;
investigating the frequency of up-regulated and down-regulated genes across individual members and groups; and
forming at least one matrix which provides a basis for investigation of expression of members of said working gene set across said set of tissue samples, thereby providing at least one potential gene target.

13. The method of claim 12, wherein said investigation includes calculating an intensity ratio of a particular gene of said working gene set, said intensity ratio calculated by comparison of a particular gene expression intensity to a calculated average intensity.

14. The method of claim 13, wherein said intensity ratio is utilized to provide information utilized to construct at least a part of at least one matrix, said matrix being comprised of a plurality of calculated ratios.
